(19) United States
(12) Patent Application Publication
(10) Pub. No.: US 2011/0289992 A1
(43) Pub. Date: Dec. 1, 2011

(54) Unturned Covered Aerated Static Pile Composting System and Method

(75) Inventor: Jan Allen, Shoreline, WA (US)

(73) Assignee: HARVEST POWER, INC., Waltham, MA (US)

(21) Appl. No.: 13/085,179

(22) Filed: Apr. 12, 2011

Related U.S. Application Data
(60) Provisional application No. 61/350,230, filed on Jun. 1, 2010.

(51) Int. Cl. C05F 11/00; C12M 1/12

(52) U.S. Cl. 71/23; 435/290.1

(57) ABSTRACT

A method of unturned aerated static pile composting includes providing organic matter having oversized particles and forming a pile of the organic matter on a surface. The surface has a plurality of compost pipes. The method further includes covering at least a portion of the pile with an organic and particulate layer and then providing air flow through the compost pipes such that a negative air pressure is formed through the pile causing air and fluid to be withdrawn from the pile into the compost pipes. The method further includes inserting a spike in the pile at designated areas and times in order to form air shafts in the pile. The composting system includes a compost enclosure surrounding a plurality of compost pipes, each compost pipe having an air flow control valve, and at least one fan, each fan having an air intake and an air outlet. The air intake is in fluid communication with the plurality of compost pipes. The system also includes a biofilter system in fluid communication with the air outlet of the fan(s) and a spike configured for insertion into the compost pile in order to form air shafts in the compost pile.

### CROSS-REFERENCE TO RELATED APPLICATIONS

The present application claims priority to U.S. Provisional Patent Application No. 61/350,230 filed Jun. 1, 2010, the disclosure of which is incorporated by reference herein in its entirety.

### TECHNICAL FIELD

The invention generally relates to composting systems and, more particularly, the invention relates to unturned covered aerated static pile composting systems.

### BACKGROUND ART

Current state of the art requires that the composting process be controlled continuously from start to finish with minimal air emissions. Air emissions are more significant during feedstock receiving, feedstock preparation, and pile turning, both teardown and rebuild, after the primary or first stage of composting. Once a pile is placed, undisturbed emissions are not zero but diminish significantly, and are considered minimal. Consequently, the material handling during feedstock preparation and pile turning causes the greatest emissions. Existing composting systems today range from outdoor systems with no emission control to those with complete enclosure and exhaust treatment devices. The regulatory trend in North America is to regulate composting emissions for odor, ammonia, volatile organic compounds (VOC), and/or greenhouse gases. This trend is causing an increase in the number of modified systems that utilize the aerated static pile technology.

Aerated static pile composting was developed in 1973 by the USDA. Generally, aerated static pile composting involves a controlled aeration method, such as a piping system under the pile or piles, and a residence time of at least 14 days. These static pile composting systems generally involve grinding and then mixing the organic feedstock materials so each organic particle is approximately 6 inches or less in its maximum dimension. However, grinding is expensive and relatively slow so organic waste materials typically accumulate in an unprocessed and odorous state if the mass rate of incoming material is greater than the grinding rate. For example, a large facility may accept over 1,000 tons per day of materials but its grinding rate might only be 60 tons per hour.

There are a number of modified aerated static pile systems being practiced to improve odor control. The use of membranes, tarps, or covers is increasing in the industry, to help limit fugitive emissions and improve moisture control. However, all of these systems still require grinding and pile turning. Because of the denseness of the feedstock material, pile depths are generally limited to between 4 feet deep and 17 feet deep.

### SUMMARY OF EMBODIMENTS

In accordance with one embodiment of the invention, a method of aerated static pile composting provides organic matter having oversized particles and forms a pile of the organic matter on a surface. The surface has a plurality of compost pipes. The method further covers at least a portion of the pile with an organic and particulate layer and then provides air flow through the compost pipes such that a negative air pressure is formed through the pile causing air and fluid to be withdrawn from the pile into the compost pipes. The method further includes inserting a spike in the pile at designated areas and times in order to form air shafts in the pile.

In accordance with related embodiments, the compost pile may be formed with a height of about 15-25 feet. An additional organic and particulate layer (cover) may be formed on top of this height. The organic matter may not undergo a particle size reduction process (grinding) prior to forming the pile. The compost pile may have a density of no greater than about 850 pounds per cubic yard with a minimum porosity of about 45% by volume. The compost pile may have a density ranging from about 650 to about 850 pounds per cubic yard. The oversized particles may include brush, branches, small stumps, dimensional wood, pallets, and/or crating. The compost pile may be formed within an enclosure that surrounds a bottom portion of the pile. The compost pipes may be placed on top of the surface or within channels formed in the surface. The organic matter may include high-carbon amendments of at least about 95% carbon. The high-carbon amendments may include cedar bark, wood, sawdust, and/or paper. The organic and particulate layer may be at least about six inches thick and may include at least one of compost, bark, wood ash, sawdust, and wood chips. The method may further include providing a biofilter in fluid communication with the compost pipes such that the air and fluid withdrawn from the pile into the compost pipes is transported to the biofilter for exhaust treatment. The method may further include taking a sample of the organic matter with the spike in order to analyze a lower portion of the pile.

In accordance with another embodiment of the invention, an aerated static pile composting system includes a compost enclosure surrounding a plurality of compost pipes, each compost pipe having an air flow control valve. The compost enclosure is configured to hold a compost pile having organic material with oversized particles. The composting system also includes at least one fan, each fan having an air intake and an air outlet. The air intake is in fluid communication with the plurality of compost pipes. The fan(s) are configured to provide air flow through the compost pipes such that a negative air pressure is formed through the compost pile causing air and fluid to be withdrawn from the compost pile and into the compost pipes. The composting system also includes a biofilter system in fluid communication with the air outlet of the fan(s) and a spike configured for insertion into the compost pile in order to form air shafts in the compost pile. The fan(s) are configured to transport the air and fluid withdrawn from the compost pile to the biofilter system.

In related embodiments, the composting system may further include a compost surface within the compost enclosure. The compost pipes may be placed on top of the surface or placed within channels formed in the surface. The spike may further include a sampling corbel on a side of the spike near its end.

### BRIEF DESCRIPTION OF THE DRAWINGS

The foregoing features of various embodiments of the invention will be more readily understood by reference to the following detailed description, taken with reference to the accompanying drawings, in which:

FIG. 1 schematically shows an aerated static pile composting system according to illustrative embodiments of the present invention;

FIG. 2 shows a process of aerated static pile composting according to embodiments of the present invention;

FIG. 3A schematically shows a side-view of an compost pipe according to embodiments of the present invention;

FIG. 3B schematically shows a cross-sectional view of the compost pipe along line A-A of FIG. 3A within a channel of a surface;

FIG. 3C schematically shows an alternative cross-sectional view of the compost pipe along line A-A of FIG. 3A within a channel of a surface;

FIG. 4 shows compost pipes on the inside of a compost enclosure according to embodiments of the present invention;

FIG. 5 shows an outside of a compost enclosure with conduits connecting the compost pipes to a compost manifold according to embodiments of the present invention;

FIG. 6 shows an airflow control valve on a conduit according to embodiments of the present invention;

FIG. 7 shows fans connecting the compost manifold to the biofilter manifold according to embodiments of the present invention;

FIG. 8A schematically shows a side-view of a biofilter pipe according to embodiments of the present invention;

FIG. 8B schematically shows a cross-sectional view of a biofilter pipe along line A-A of FIG. 8A within biofilter material;

FIG. 9 shows biofilter pipes within a biofilter enclosure according to embodiments of the present invention;

FIG. 10 schematically shows an illustrative biofilter system that may be used with embodiments of the present invention;

FIG. 11A shows a spike attached to a machine according to embodiments of the present invention;

FIG. 11B shows a plan view of a portion of the spike with a sampling corbel according to embodiments of the present invention; and

FIG. 11C shows a side-view of the sampling corbel shown in FIG. 11B.

### DESCRIPTION OF ILLUSTRATIVE EMBODIMENTS

Various embodiments of the present invention provide an aerated static pile composting system and method that includes larger, oversized particles and eliminates the need for initial grinding of the feedstock material. Brush, branches, small stumps, dimensional wood, pallets, and grating may be directly and immediately placed into the process without particle size reduction allowing for more rapid feedstock receiving and preparation. In addition, embodiments eliminate the need for pile turning during the composting process. As a result, emissions are significantly reduced. Because the un-ground feedstock is lower in bulk density and higher in the porosity due to the inclusion of the larger particles, a deeper composting pile may be used. Thus, embodiments of the present invention provide more space efficiency than other covered aerated static pile systems (e.g., approximately 105,000 metric tonnes/year/hectare versus 24,000 metric tonnes/year/hectare for others) and allow for more seasonal composition, volume, and moisture variations through the use of a deeper composting pile and the addition of high-carbon amendments in the pile. Details of illustrative embodiments are discussed below.

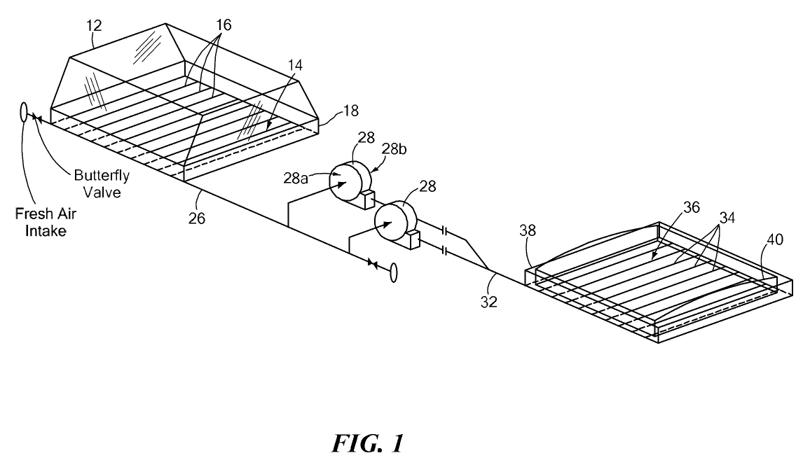

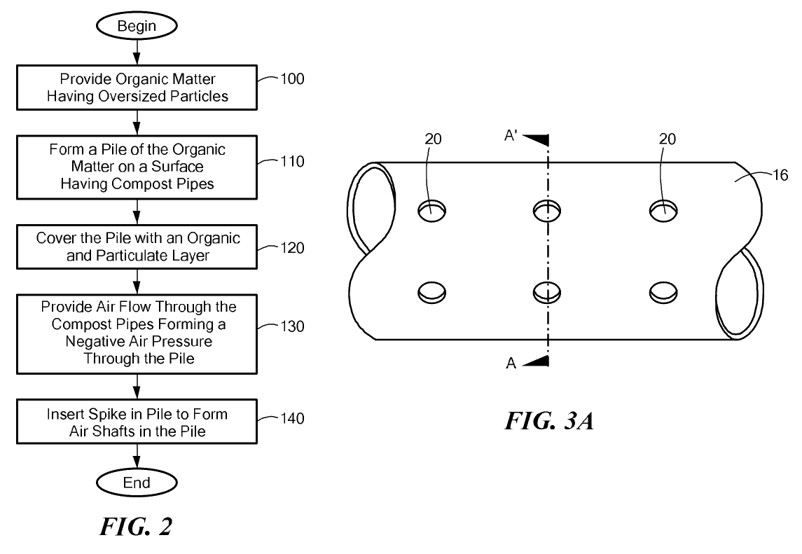

FIG. 1 schematically shows an aerated static pile composting system and FIG. 2 shows a process of aerated static pile composting according to embodiments of the present invention. Referring to FIG. 2, the process begins at step 100, in which organic matter having oversized particles is provided. The oversized particles may include brush, branches, small stumps, dimensional wood, pallets, and/or crating. Because oversized particles are used, initial grinding of the feedstock is eliminated which allows for more rapid feedstock receiving and preparation. The use of the oversized particles, along with a pile restructuring apparatus, also eliminates the need for pile turning during the composting process. The pile restructuring apparatus or spike is discussed in more detail below. During periods when larger particle materials are unavailable, the feedstock may be amended with screening oversized material, large woody particles cast off in the screening process, bark, and similar forest product residuals. The removal of the oversized particles may be accomplished at the final screening process after the composting process is complete. Preferably, the feedstock is a mixture of incoming organic matter, screening oversized particles and woody materials. The feedstock also, preferably, includes high-carbon amendments of at least about 95% carbon. The high-carbon amendments may include cedar bark, wood, sawdust and/or paper.

Referring also to FIG. 1, in step 110, a compost pile 12 of the organic matter is formed on a surface 14 that has a series of compost pipes 16. A compost enclosure 18 surrounds the surface 14 and is configured to hold the compost pile 12. Preferably, the compost pile 12 is initially formed with a height or cross-section of at least about 20 feet. This deeper cross-section is possible due to the use of the un-ground feedstock which is lower in bulk density, e.g., about 650 to about 850 lbs/cubic yard compared to about 850 to about 950 lbs/cubic yard commonly used in practice today. For example, for a compost pile 12 having a height of about 25 feet, the feedstock density is preferably about 750 lbs per cubic yard with a minimum porosity of about 45% by volume, which equates to about 45 to about 50% solids.

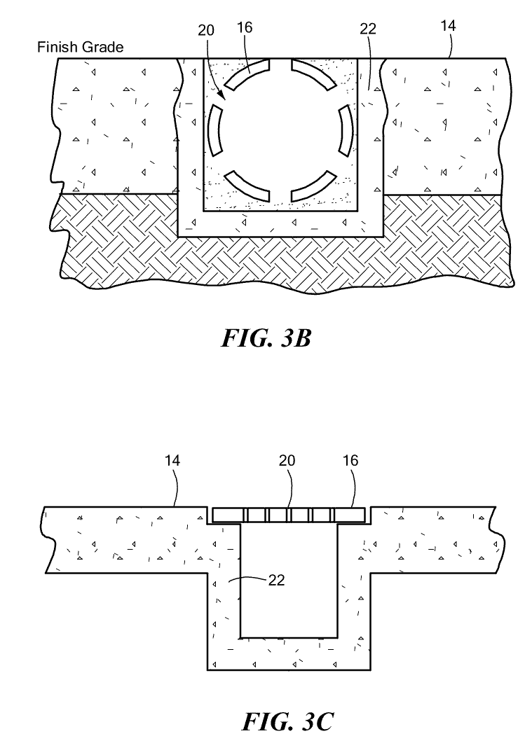

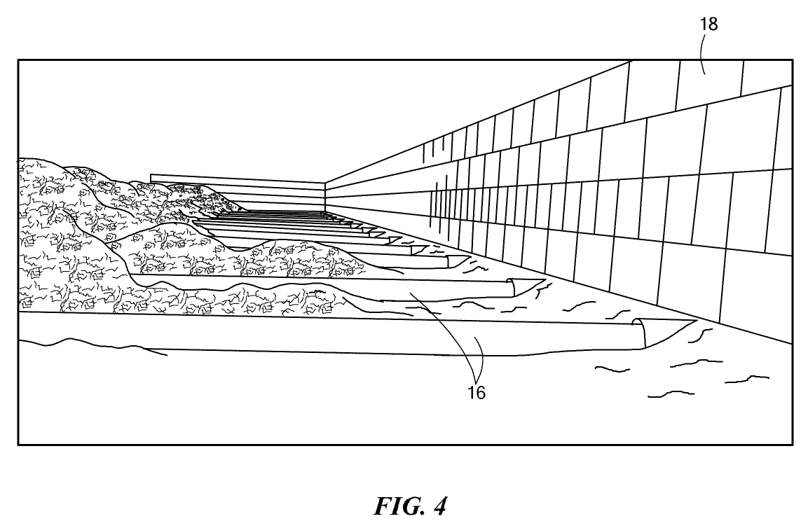

FIGS. 3A, 3B and 3C schematically show a side-view and cross-sectional views, respectively, of one illustrative compost pipe 16. Each of the compost pipes 16 has holes 20, such as shown in FIGS. 3A through 3C, that allow fluid to flow from an area outside of the pipe 16 to within the pipe 16. Each of the compost pipes 16 may be placed on top of the surface 14 or may be placed within channels 22 formed within the surface 14, such as shown in FIGS. 3B and 3C. The compost pipes 16 may be made from perforated extra heavy duty pipe, such as high density polyethylene, so that the pipes 16 may be used on a soil or pavement surface. For example, each pipe may have an outside diameter of about 12.75 inches, an inside diameter of about 10.3 inches, and a wall thickness of about 1.16 inches. Preferably, the holes 20 in the pipe 16 have about a 2 inch diameter when used on pavement surfaces and have about a 0.75 inch diameter when used on soil. The series of compost pipes 16 may be spaced any distance apart from one another, e.g., about 17 feet apart. The compost pipes 16 may have stainless steel couplings to allow pipe section removal and replacement without the need for thermal fusion equipment. As shown in FIG. 4, the compost pipes 16 may run underneath the compost enclosure 18 or may run through openings formed in the compost enclosure 18 (not shown).

Referring again to FIG. 2, in step 120, the compost pile 12 is covered with an organic and particulate layer. This cover prevents fugitive odor release caused by convection, heat rise, and draft induced by ambient wind on the compost pile 12. Wind tends to strip odors out of the windward side and top edge of the pile 12. Preferably, the cover is approximately 6 inches thick and includes compost, bark, wood ash, sawdust, and/or wood chips. The cover may be placed after the compost pile 12 is constructed, left in place during the composting process, and then removed for reuse before the pile 12 is torn down for screening.

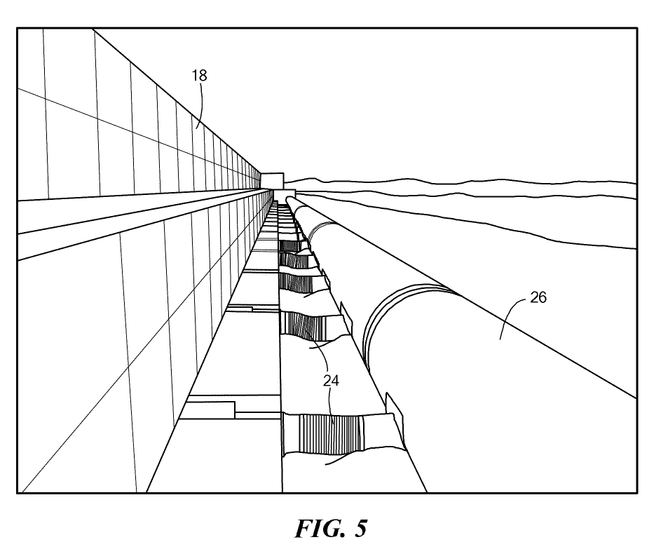

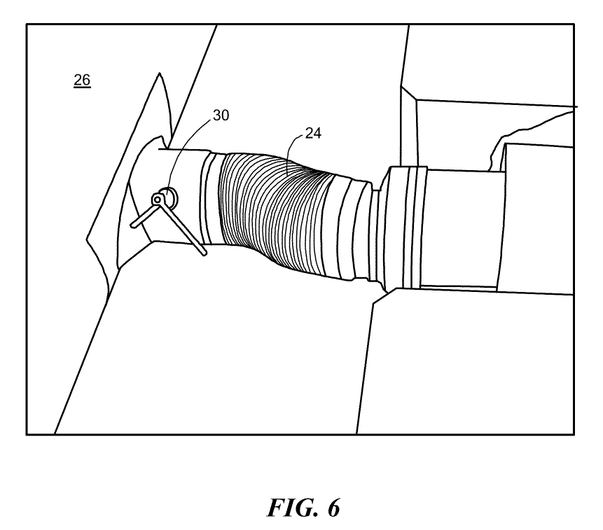

In step 130, air flow is provided through the compost pipes 16 such that a continuous low-rate negative pressure system is formed in the pile causing air and fluid to be withdrawn from the pile into the compost pipes 16. Referring also to FIG. 5, each compost pipe 16 is in fluid communication with a conduit 24 which is in fluid communication with a compost manifold 26. The compost manifold 26 is also in fluid communication with one or more fans 28 and is connected to the air intake side 28a of the fans 28.

When the fans 28 are operational, air flows through the compost manifold 26 and the inner portion of the compost pipes 16 forming an induced vacuum below the compost pile 12. The negative air pressure through the compost pile 12 causes air and fluid to be withdrawn from the pile 12 through the holes 20 and into the compost pipes 16. The amount of fluid flow through each compost pipe 16 may be regulated by varying the speed of the fans 28 or may be regulated by an airflow control valve 30 on each of the conduits 24, such as shown in FIG. 6. The control valves 30 may be manually or automatically controlled.

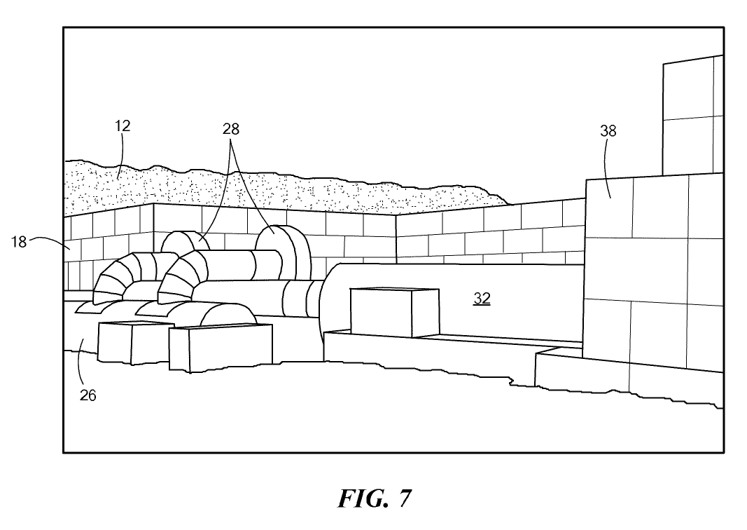

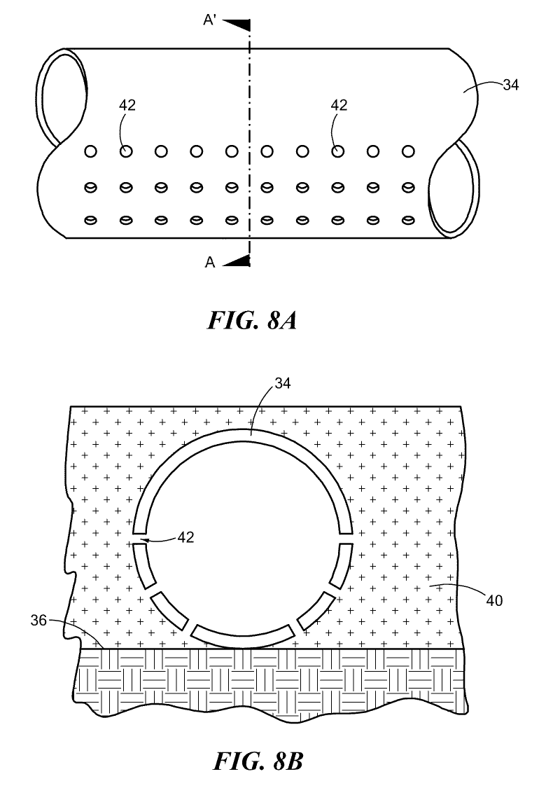

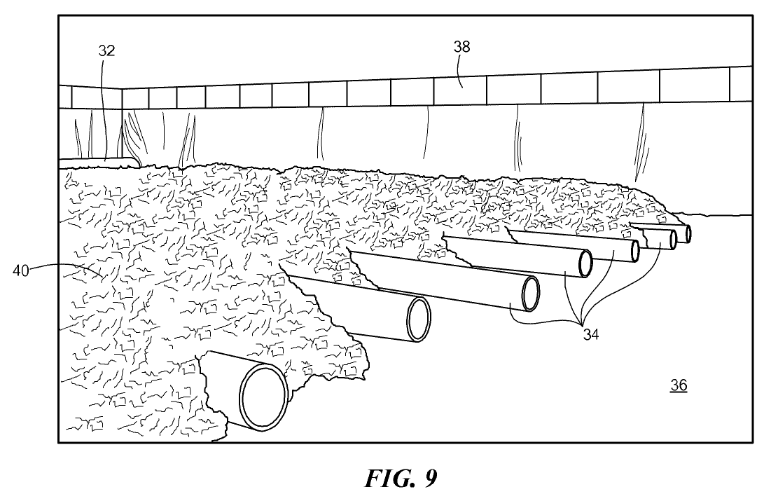

The air and fluid taken from the compost pile 12 is then transported to and discharged into a biofilter system for odor control. This is accomplished by the air and fluid flowing through the compost manifold 26 and fans 28 and into a biofilter manifold 32, which is in fluid communication with the one or more fans 28 and connected to the air output side 28b of the fans 28. FIG. 7 shows one embodiment of the fans 28 connecting the compost manifold 26 to the biofilter manifold 32. The biofilter manifold 32 is also in fluid communication with a series of biofilter pipes 34, which are disposed on or in a biofilter surface 36 surrounded by a biofilter enclosure 38. The biofilter manifold 32 may run through an opening formed in the biofilter enclosure 38. The biofilter enclosure 38 is configured to hold biofilter media 40 formed around and on top of the biofilter pipes 34. FIGS. 8A and 8B schematically show a side-view and cross-sectional view, respectively, of one illustrative biofilter pipe 34. Each of the biofilter pipes 34 has holes 42, such as shown in FIGS. 8A and 8B, that allow fluid to flow from within the biofilter pipe 34 to an area outside of the pipe 34 which contains the biofilter media 40. Each of the biofilter pipes 34 may be placed on top of the biofilter surface 36, such as shown in FIG. 8B, or may be placed within channels (not shown) formed within the biofilter surface 36. FIG. 9 shows one embodiment of biofilter pipes 34 placed on top of a biofilter surface 36 and surrounded by biofilter media 40.

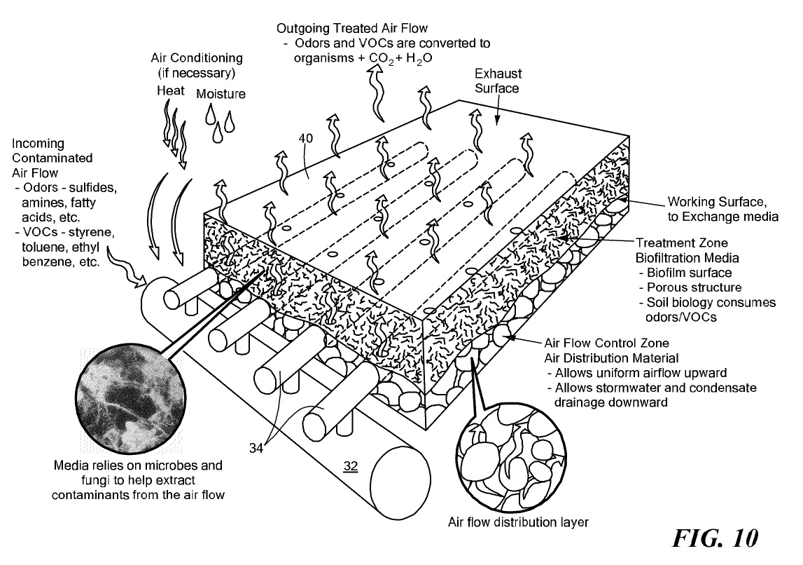

As known by those skilled in the art, the biofilter media 40 may be composed of various materials and layers, such as shown in FIG. 10. For example, the biofilter media may include shredded wood and bark, preferably about 75% wood and about 25% bark. Other acceptable green materials may include plant leaves, needles, and grass, although preferably these are no more than about 2% by wet weight of the biofilter media. Dimensional wood, stumps, trees, clean plywood, and clean particle board may also be used. Preferably, the biofilter media 40 includes at least about 60% organic matter, a maximum TKN nitrogen of no more than 0.35%, a moisture content of between about 35 to about 60%, and combined nitrate and ammonium concentrations that are less than about 100 ppm. The biofilter media 40 also preferably includes at least about 90% by weight of particle sizes ranging from about 1.0 to about 4.0 inches, with less than about 10% by weight of particle sizes ranging less than about 1.0 inch and less than about 5% by weight of particle sizes ranging greater than about 4.0 inches.

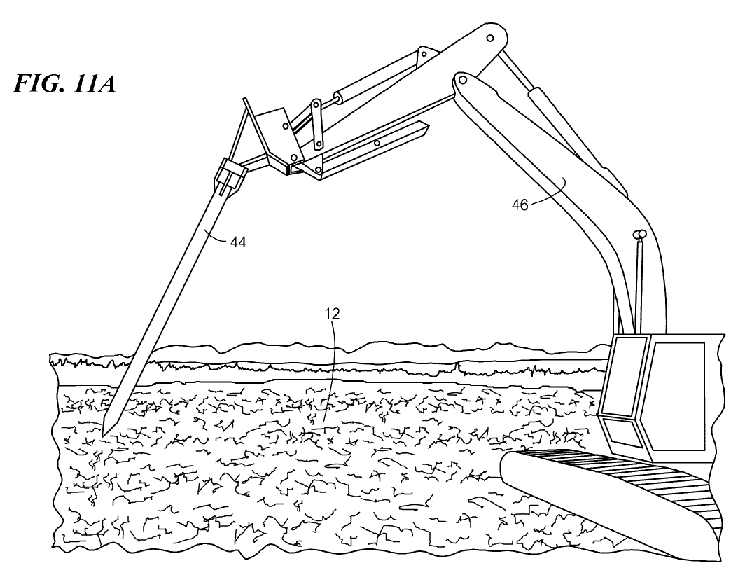

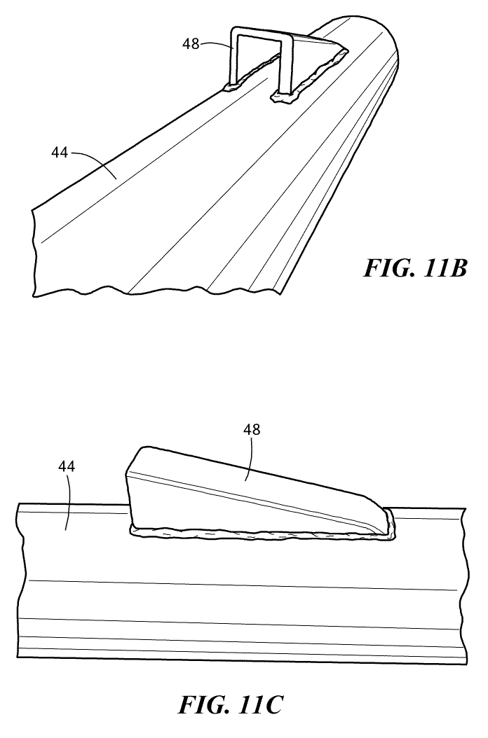

Referring again to FIG. 2, in step 140, a pile restructuring apparatus or spike 44 is inserted into the compost pile 12 after a designated period of time in order to form air shafts in the pile 12. The air shafts repair uneven airflow allowing substantially uniform aerobic conditions in the compost pile 12 for much longer periods of time (e.g., 30-65 days compared to 15-30 days commonly used in practice today). As shown in FIGS. 11A-11C, the spike 44 has a long shaft mounted on a machine 46, such as an excavator, and includes a sampling corbel 48 attached on a side of the spike 44 toward its end. In operation, the machine 46 moves across the top of the compost pile 12 and punctures the pile 12 with the spike 44 at designated areas leaving vertical air shafts throughout the pile 12. The air shafts may be formed in a uniform array of shafts across the pile 12 or in an uneven pattern, e.g., in designated areas where more aerobic conditions are needed. For example, the air shafts may be spaced about 8 feet apart from the center of one shaft to the center of another. Preferably, the spike 44 is long enough so that the air shafts are formed through at least half the height of the pile 12. For example, for a compost pile 12 having an initial height of about 25 feet, the spike 44 may be about 13 feet long and have about an 8 inch diameter. The sampling corbel 48 allows a small sample of the lower horizon of the pile 12 to be brought to the surface for observation and mapping of the lower horizon. The inspection of the sample may include a visual inspection of the moisture, color, texture, odor, and/or temperature of the organic matter. The observations and mapping may be recorded. This information may then be used to individually adjust airflow up or down each pipe, or may be used to increase or decrease the airflow in general for the whole composting system. Forming the array of air shafts across the compost pile 12 with the spike 44 may be done one or more times during the composting process, preferably about one to four times for a compost pile 12 having a composting process of about two months. The use of the spike 44 allows the organic matter in the compost pile 12 to have sufficient aerobic conditions for the composting process without the need for turning (tearing down and rebuilding) the compost pile 12.

Although the above discussion discloses various exemplary embodiments of the invention, it should be apparent that those skilled in the art can make various modifications that will achieve some of the advantages of the invention without departing from the true scope of the invention.

What is claimed is:

1. A method of aerated static pile composting, the method comprising:
providing organic matter having oversized particles;
forming a pile of the organic matter on a surface, the surface having a plurality of compost pipes;
covering at least a portion of the pile with an organic and particulate layer;
providing air flow through the compost pipes such that a negative air pressure is formed through the pile causing air and fluid to be withdrawn from the pile into the compost pipes; and
inserting a spike in the pile at designated areas and times in order to form air shafts in the pile.

2. The method of claim 1, wherein the pile is formed with a height of at least about 20 feet.

3. The method of claim 1, wherein the organic matter does not undergo a particle size reduction process prior to forming the pile.

4. The method of claim 1, wherein the pile has a density of no greater than about 850 pounds per cubic yard with a minimum porosity of about 45% by volume.

5. The method of claim 4, wherein the pile has a density ranging from about 650 to about 850 pounds per cubic yard.

6. The method of claim 1, wherein the oversized particles include brush, branches, small stumps, dimensional wood, pallets, crating or a combination thereof.

7. The method of claim 1, wherein the pile is formed within an enclosure that surrounds a bottom portion of the pile.

8. The method of claim 1, wherein the compost pipes are placed on top of the surface.

9. The method of claim 1, wherein the compost pipes are placed within channels formed in the surface.

10. The method of claim 1, wherein the organic matter includes high-carbon amendments of at least about 95% carbon.

11. The method of claim 10, wherein the high-carbon amendments include cedar bark, wood, sawdust, paper, or a combination thereof.

12. The method of claim 1, wherein the organic and particulate layer includes at least one of compost, bark, wood ash, sawdust, and wood chips.

13. The method of claim 1, wherein the organic and particulate layer is at least about six inches thick.

14. The method of claim 1, further comprising providing a biofilter in fluid communication with the compost pipes, wherein the air and fluid withdrawn from the pile into the compost pipes is transported to the biofilter.

15. The method of claim 1, further comprising taking a sample of the organic matter with the spike in order to analyze a lower portion of the pile.

16. A aerated static pile composting system comprising:
a compost enclosure surrounding a plurality of compost pipes and configured to hold a compost pile having organic material with oversized particles, each compost pipe having an air flow control valve;
at least one fan, each fan having an air intake and an air outlet, the air intake in fluid communication with the plurality of compost pipes, the at least one fan configured to provide air flow through the compost pipes such that a negative air pressure is formed through the compost pile causing air and fluid to be withdrawn from the compost pile and into the compost pipes;
a biofilter system in fluid communication with the air outlet of the at least one fan, the at least one fan configured to transport the air and fluid withdrawn from the compost pile to the biofilter system; and
a spike configured for insertion into the compost pile in order to form air shafts in the compost pile.

17. The aerated static pile composting system of claim 16, further comprising a compost surface within the compost enclosure, wherein the compost pipes are placed on top of the surface.

18. The aerated static pile composting system of claim 16, further comprising a compost surface within the compost enclosure, wherein the compost pipes are placed within channels formed in the surface.

19. The aerated static pile composting system of claim 16, wherein the spike further includes a sampling corbel on a side of the spike near its end.
